(19) United States
(12) Patent Application Publication
(10) Pub. No.: US 2013/0345623 A1
(43) Pub. Date: Dec. 26, 2013

(54) DEVICE AND METHOD FOR CREATION AND DISPLAY OF GRAPHIC CODING SPECIFIC FOR MEDICAL DEVICES AND MEDICAL TREATMENTS

(71) Applicant: Fresenius Medical Care Deutschland GmbH, Bad Homburg (DE)

(72) Inventors: Pascal KOPPERSCHMIDT, Dittelbrunn (DE); Carsten MUELLER, Euerbach (DE); Matthias PFEUFFER, Ebenhausen (DE)

(73) Assignee: Fresenius Medical Care Deutschland GmbH, Bad Homburg (DE)

(21) Appl. No.: 13/924,922

(22) Filed: Jun. 24, 2013

Related U.S. Application Data
(60) Provisional application No. 61/663,159, filed on Jun. 22, 2012.

(30) Foreign Application Priority Data
Jun. 22, 2012 (DE) 102012012350.7

(51) Int. Cl. A61M 1/14; G06K 7/01; G06K 19/06; A61M 1/28; A61M 1/34

(52) U.S. Cl. CPC A61M 1/14; A61M 1/28; A61M 1/34; G06K 19/06009; G06K 7/01
USPC 604/29; 235/494; 235/435; 235/375

(57) ABSTRACT

The present invention relates to the field of information display and forwarding with medical machines, in particular display and forwarding of information pertaining to the medical machine such as treatment information, equipment information and software codes. The invention is based on the problem of providing the patient with information about his/her treatment in a simple and convenient manner and/or providing the operator or service personnel of the medical machine information pertaining to the equipment of the medical machine and/or its software options in a simple and convenient manner. A method and a device in which characteristic graphic codes are generated and displayed and which can be input and decoded by a portable device are proposed for this purpose.

### TECHNICAL FIELD

The invention relates to the field of devices and methods for creating and display of graphic coding specific for medical devices and medical treatments, in particular barcodes and two-dimensional graphic codes.

### PRIOR ART

Medical devices are understood in particular to also include fluid treatment devices such as blood treatment devices in which fluid from a patient is supplied through a fluid line to a fluid treatment component, is treated by the fluid treatment component and can be divided into an arterial branch and a venous branch through the fluid line and then returned to the patient. Examples of such blood treatment devices include in particular hemodialysis machines. One such blood treatment device is the subject matter of DE 198 49 787 C1 of the present patent applicant.

Dialysis is a method for purifying the blood of patients with acute or chronic renal insufficiency. Essentially a distinction is made here between methods having an extracorporeal blood circulation such as hemodialysis, hemofiltration or hemodiafiltration (summarized below under the heading “hemodialysis”) and peritoneal dialysis which does not use an extracorporeal blood circulation.

In hemodialysis, the blood is passed through the blood chamber of a dialyzer in an extracorporeal circulation, this blood chamber being separated from the dialysis fluid chamber by a semipermeable membrane. Dialysis fluid containing the blood electrolytes in a certain concentration flows through the dialysis fluid chamber. The substance concentration of the dialysis fluid corresponds to the concentration in the blood of a healthy person. During the treatment the blood of the patient and the dialysis fluid are passed by both sides of the membrane, usually in countercurrent at a predetermined flow rate. The substances that must be eliminated in the urine diffuse through the membrane from the blood chamber into the chamber for the dialysis fluid, while electrolytes present in the blood and in the dialysis fluid at the same time diffuse from the chamber of the higher concentration to the chamber of the lower concentration. If a pressure gradient is built up on the dialysis membrane from the blood side to the dialysate side, for example, by a pump which withdraws dialysate from the dialysate circulation on the dialysate side downstream from the dialysis filter, water is transferred from the patient's blood through the dialysis membrane and into the dialysis circulation. This method of ultrafiltration leads to the desired withdrawal of water from the patient's blood.

In hemofiltration, a transmembrane pressure is applied in the dialyzer to remove ultrafiltrate from the patient's blood without passing the dialysis fluid by the side of the membrane of the dialyzer opposite the patient's blood. In addition a sterile and pyrogen-free substituate solution may be added to the patient's blood. Depending on whether this substituate solution is added upstream from the dialyzer or downstream, we speak of pre- or post-dilution. The mass exchange in hemofiltration takes place by convection.

Hemodiafiltration combines the methods of hemodialysis and hemofiltration. A diffusive mass exchange takes place between the patient's blood and the dialysis fluid through the semipermeable membrane of a dialyzer and plasma water is also filtered through a pressure gradient on the membrane of the dialyzer.

Plasmapheresis is a method by which the patient's blood is separated into the blood plasma and its corpuscular components (cells). The blood plasma separated is purified or replaced by a replacement solution and returned to the patient.

In peritoneal dialysis a patient's abdominal cavity is filled through a catheter passed through the abdominal wall with a dialysis fluid having a concentration gradient with respect to the endogenous fluids. The toxins present in the body enter the abdominal cavity through the peritoneum which acts as a membrane. After a few hours the dialysis fluid, now spent, which is in the patient's abdominal cavity is replaced. Water can be removed from the patient's blood through the peritoneum and can enter the dialysis fluid and thereby withdraw water from the patient.

The methods of dialysis are usually performed with the help of automatic dialysis machines such as those distributed by the present applicant under the brand name 5008 or sleep.safe.

These dialysis machines are complex medical devices with extensive functions. Such machines are often operated by touchscreen displays which are to be understood as combined input and output devices.

Patients suffering from renal insufficiency must undergo dialysis treatments regularly. The success of these treatments depends on many parameters.

To provide the treating physicians with a return message about the success of the treatment, a number of parameters can be monitored and recorded and/or stored during the treatment.

These parameters include, for example, the total volume of water withdrawn from the patient during a dialysis treatment, the quantity of substituate added to the patient's blood during a dialysis treatment, vital statistics, such as blood pressure, body temperature and pulse rate before, during and at the end of a treatment, the type of medical articles used such as tube sets, dialysis filters, dialysis fluid, substituate or electric conductivity values in the blood or dialysate.

Other parameters can be monitored and recorded accordingly for other medical devices and treatments.

The parameters recorded are usually in the form of electronic data in a memory provided for this purpose. The treating physician can have access to these data in various ways. For example, these data can be transferred to a remote computer to which the treating physician has access.

Systems in which treatment-related data are stored on a patient card which has its own data memory are also known. The physician will then have a card reader with the help of which he can read out the stored treatment-related data from the patient card.

In this way, the treating physician is informed about the success of the treatments that have been performed and can make a recommendation for the parameters for the next pending treatment. Such a recommendation is then called a prescription.

As a rule the patient himself will not have a reader for the patient card or access to the data stored during the treatment. The storage and processing of the treatment-related data are also directed at medically trained personnel. A patient usually does not have the medical expertise to interpret the data processed on such a card.

One object of the present invention is therefore to create a device and a method with which a patient can be informed easily and conveniently about the course of his treatments.

Another object achieved with the device and the method according to the invention is convenient activation of software updates of medical devices.

Medical devices, in particular automatic dialysis machines are complex technical devices which are often microprocessor-controlled. Such microprocessor-controlled devices are based on software installed in them which programs the microprocessors. The interaction between software and hardware here defines the functionality of the medical device.

A medical device may be supplied with different versions of software to enable different types of treatment, for example. In addition, the software can be updated regularly to allow improvements or new options to be incorporated.

As a rule the user must pay for the use of improved or expanded software versions. Technically the acquisition of new software for medical devices is often regulated by having the various software versions and/or software options available, for example, as a program reserved for downloading on a server and/or by distributing storage media such as CDs or USB sticks.

The user of a medical device can install the new software in the medical device, for which purpose interfaces such as network connections may be provided on the medical device.

To finally install the new software and/or the new software version, after which the medical device is controlled according to the programming of the new software, it is often necessary to enter an unlocking key into the medical device.

This unlocking key serves to ensure that the software installed has been licensed, i.e., paid for properly.

To obtain the unlocking key, an alphanumeric code is output after installing the new software, and this code is then usually displayed on the screen of the medical device.

The user records this alphanumeric code and supplies it to the software provider, e.g., by email. The alphanumeric code is characteristic of the new software and may also code for additional information such as the model number or serial number of the medical device or the identity of the purchaser or the location of the medical device.

The software provider determines the purchase price to be charged from the alphanumeric code transmitted. The software provider can then recognize, on the basis of the payments entered, whether the software installed has been properly licensed, i.e., paid for.

Only after this has been confirmed does the software provider in turn generate a characteristic license key, which in turn is transmitted to the user by email for example. To unlock the software the user must enter this license key into the medical device, for example, by manually typing it in a field provided for this purpose on the touchscreen.

The alphanumeric codes, i.e., license keys often consist of a plurality of numbers and characters. Manual reading and transfer of these codes is therefore inconvenient and subject to error.

Therefore, another object of the present invention is to make the activation of software installed in medical devices more convenient and more reliable.

Another aspect of the present invention relates to the display of information pertaining to the medical device in a service event. In the event a service operation is required, for example, for maintenance or repair of the medical device or in the event of a defect, it is often necessary to know the exact type of medical device including all accessory parts and software versions and to transmit it to the service personnel in advance.

This information has so far been displayed in plain text and must be copied by the user in a tedious operation and transmitted. This is inconvenient and susceptible to error.

Therefore another object of the present invention is to make the transmission of information pertaining to medical devices more convenient and more reliable.

### DETAILED DESCRIPTION OF THE INVENTION

Consequently, the objects on which the present invention is based are to create a device and a method by which a patient can be informed easily and conveniently about the course of his treatments and with which the activation of software installed in medical devices and the transmission of information pertaining to medical devices can be made more convenient and more reliable.

These objects are achieved according to the invention by a method having the features of claim 1 and by a device having the features of claim 9.

Advantageous embodiments are the subject matter of the dependent claims.

The invention is thus based on a method for displaying information pertaining to a medical device, this method comprising the steps of generating a graphic code which is characteristic of information pertaining to the medical device and display of the graphic code.

In addition, the invention is directed at a medical device having a display screen and a control unit, wherein the control unit is equipped to generate a graphic code characteristic of the information pertaining to the medical device and to display this code on the display screen.

A medical device is understood to include dialysis machines in particular. The present invention is to be explained below on the example of a dialysis machine equipped for hemodialysis. It should be clear to those skilled in the art that this invention can be used with a variety of medical devices.

Information pertaining to the medical device includes in the sense of the invention both treatment-specific data, in particular recorded measured values, such as the blood flow rate, dialysis flow rate or volumes, such as the ultrafiltration volume and the substituate volume accumulated via the therapy, as well as data pertaining to the equipment and/or instrumentation and the condition of the medical machine. These include, for example, device and/or machine identification numbers, network addresses, Mac addresses, software version numbers, operating time of the machine, location of the machine, error messages, built-in accessories, connected accessories such as tube sets, bags, filters, drip chambers and the like.

A dialysis machine is often equipped with a display device. The display device often comprises a display which may also be embodied as a touchscreen display. Such a touchscreen display combines in one common surface an input device and an output device, in which it supplies a touch-sensitive surface with which operator input can be detected.

However, spatial separation of the input device and the output device is also conceivable, for example, embodied as a conventional display, for example, as a CRT monitor, LCD, plasma display or OLED display, as an output device and a spatially separate touchpad, which provides a touch-sensitive surface with which operator input can be detected as the input device.

Medical devices such as dialysis machines often have at least one control unit. Such a control unit controls the components of the medical device on the basis of their parameter values that characterize the medical device and are known to the control unit, among other things. Such control units are often equipped with programmable microprocessors or microcontrollers, wherein the programs controlling them are stored in program data memories provided for this purpose.

Multiple control units are often also provided in one medical device. A control unit in the sense of this invention is also understood to be a plurality of control units.

In addition medical devices such as dialysis machines often have transmission and reception equipment, where transmission and reception equipment is understood to refer to all devices with which data, in particular digital data can be sent to a remote device and/or received from a remote device. These include digital interfaces in particular, such as network interfaces which may be hardwired (for example, RJ45) as well as wireless (for example, WLAN, Bluetooth, infrared or UMTS).

In addition, medical devices may also comprise input devices for input of graphic information such as images or barcodes. Such input devices may be scanners or cameras.

A patient who undergoes a dialysis treatment is often suffering from chronic renal failure. This means that such a patient must undergo regular treatments (every two to three days), which may last several hours per treatment.

During the dialysis treatment, the patient's blood is purified and excess water is removed from the patient's blood. In the past, no feedback about the course of the treatment has been made available explicitly for the patient. With the help of the present invention, however, explicit feedback about the course of the treatment may be sent to the patient.

Such feedback may inform the patient about any change in treatment-related parameters during dialysis, for example. These parameters include, for example, the total volume of water withdrawn from the patient during a dialysis treatment, the quantity of substituate added to the patient's blood during a dialysis treatment, vital values such as blood pressure, body temperature and pulse rate before, during and at the end of a treatment, the type of medical articles used such as the tube set, dialysis filter, dialysis fluid, substituate or electric conductivity values in the blood or dialysate.

This information is coded in a graphic code and displayed on the display device of the dialysis machine after the end of the treatment. Such a graphic code may be a barcode, for example, or a so-called QR code, which is more complex than a conventional barcode and consequently offers more extensive coding options. The QR code (Quick Response) is a two-dimensional code. Other two-dimensional codes include the Micro-QR code, the Secure-QR code (SQRC) and the iQR code.

The patient enters this graphic code using a portable device. The portable device therefore has a corresponding input device such as a camera or a scanner. In addition the portable device comprises a control unit which is capable of decoding the graphic code input. To do so, the control unit, which usually comprises a microprocessor, is programmed by corresponding software. As a rule, the portable device is equipped with at least one transmission and reception device, which is understood to include all devices for sending and receiving data. This includes in particular digital interfaces such as network interfaces which may be hardwired (for example, RJ45) as well as wireless (for example, WLAN, Bluetooth, infrared or UMTS, mobile radio).

Such a portable device is especially advantageously a smartphone. Smartphones are often equipped with cameras and have a display. In addition smartphones are freely programmable by means of so-called applications. Smartphones have recently gained popularity as mobile telephones, so it is likely that a large number of dialysis patients already have such a device or will have such a device in the foreseeable future.

Due to the standardized operating systems of smartphones, corresponding programs (apps) can be provided and made available to the dialysis patients.

With a smartphone equipped in this way, the patient can enter the graphic code displayed following his treatment by photographing the code with the camera provided in the smartphone, for example.

Suitably designed software in the smartphone decodes the graphic code entered and processes the information thereby acquired further.

Further processing of this information may include display thereof. For example, the corresponding treatment-related parameters may be displayed in table form. In addition these parameters may also be stored so that a history of all the treatments completed so far is available. The course of treatment-related parameters can be displayed graphically in this way.

The possibilities of further processing of the information thereby acquired are limited only by the technical possibilities of the portable device. For example, the smartphone can also send the information to a remote device, for example, to a server, which offers access to this information for the physician treating the patient, for example.

Instead of a smartphone, a tablet PC may also be used as a portable device or any portable device which is equipped for this task and is and/or will be made available to the patient may also be used. Portable devices designed and manufactured specifically for this purpose are also conceivable.

Another aspect of the present invention relates to the unlocking of software options in the medical device. As already described above, in the past the user, i.e., the technical personnel had to manually transfer alphanumeric codes repeatedly for unlocking new software, but this is inconvenient and subject to error.

The present invention provides that this alphanumeric code is instead designed as a graphic code, which may be a barcode or a QR code.

Like the procedure followed by the patient after the treatment, the service personnel may also enter the graphic code using a suitably programmed portable device which here again is especially advantageously a smartphone.

Corresponding software in the smartphone decodes the graphic code that has been entered and then processes the information thereby obtained.

Further processing of the information may include a display of the alphanumeric code on a display screen.

Further processing of the information may include sending the alphanumeric code to a third device. The third device may be, for example, a server to which the software provider has access. This may be accomplished, for example, by sending an email.

Similarly, information pertaining to the equipment or the condition of a medical device can be characterized, displayed and processed further by using a graphic code.

### BRIEF DESCRIPTION OF THE FIGURES

Additional details and advantages of the invention are described in greater detail on the basis of exemplary embodiments shown in the figures, in which:

FIG. 1 shows a medical device according to the invention from a design as a dialysis machine, for example;

FIG. 2 shows an embodiment of the screen content of a medical device according to the invention;

FIG. 3 shows another embodiment of the screen content of a medical device according to the invention;

FIG. 4 shows an embodiment of a portable device according to the invention, embodied here as a smartphone, with screen content as an example;

FIG. 5 shows a symbolic representation of a system according to the invention comprising a medical device, a portable device and a remote device, and

FIG. 6 shows a symbolic representation of a graphic code displayed by a medical device according to the invention as well as the information, which is decoded from this graphic code and pertains to the equipment and the condition of the medical device.

### DETAILED DESCRIPTION OF FIGURES

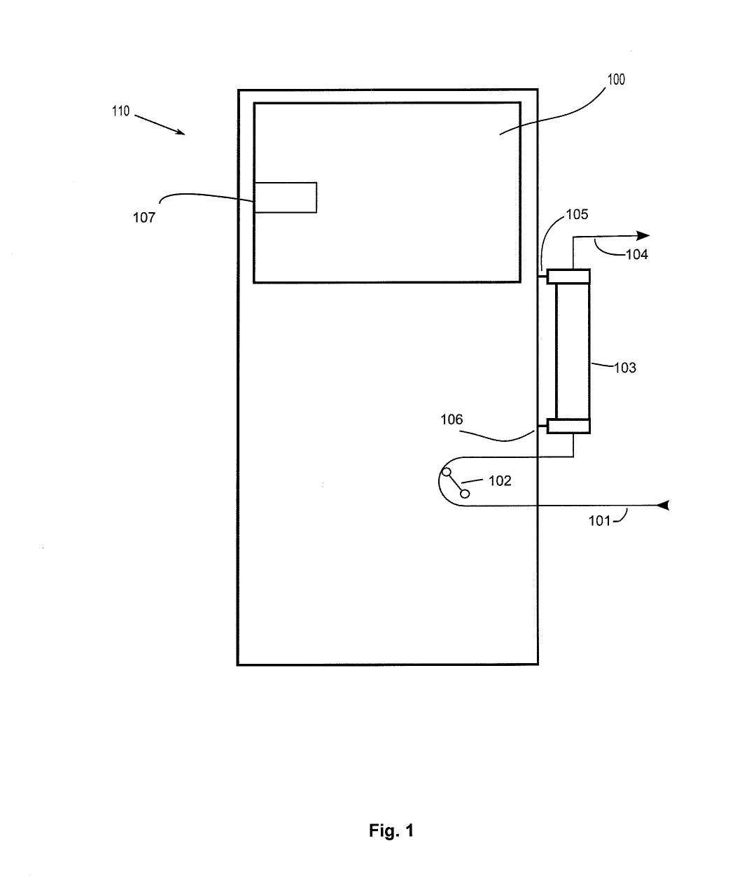

FIG. 1 shows schematically an embodiment of a medical device 110 according to the invention as a hemodialysis machine having a touchscreen display 100. The dialysis machine 110 shows parts of an extracorporeal blood circulation having an arterial bloodline 101, which draws the blood of a patient (not shown). The blood pump 102 delivers the blood through a dialysis filter 103, which is equipped with a semipermeable membrane that separates the extracorporeal blood circulation from the dialysate circulation. The treated blood is returned to the patient through the venous line 104. Dialysate is pumped through the dialysate lines 105 and 106 and through the dialysis filter 103, where it comes into diffusive mass exchange contact with the blood of a patient through the semipermeable membrane of the dialysis filter 103. If a pressure gradient is additionally built up from the blood side of the dialysis filter to the dialysate side of the patient, plasma water is expressed out of the blood into the dialysate. The expressed plasma water is also known as ultrafiltrate. Water can thus be withdrawn from the patient's blood in this way. The dialysate is prepared in the hemodialysis machine 110 and discarded after use. The touchscreen display 100 has a partial area 107, where the graphic codes according to the invention can be displayed.

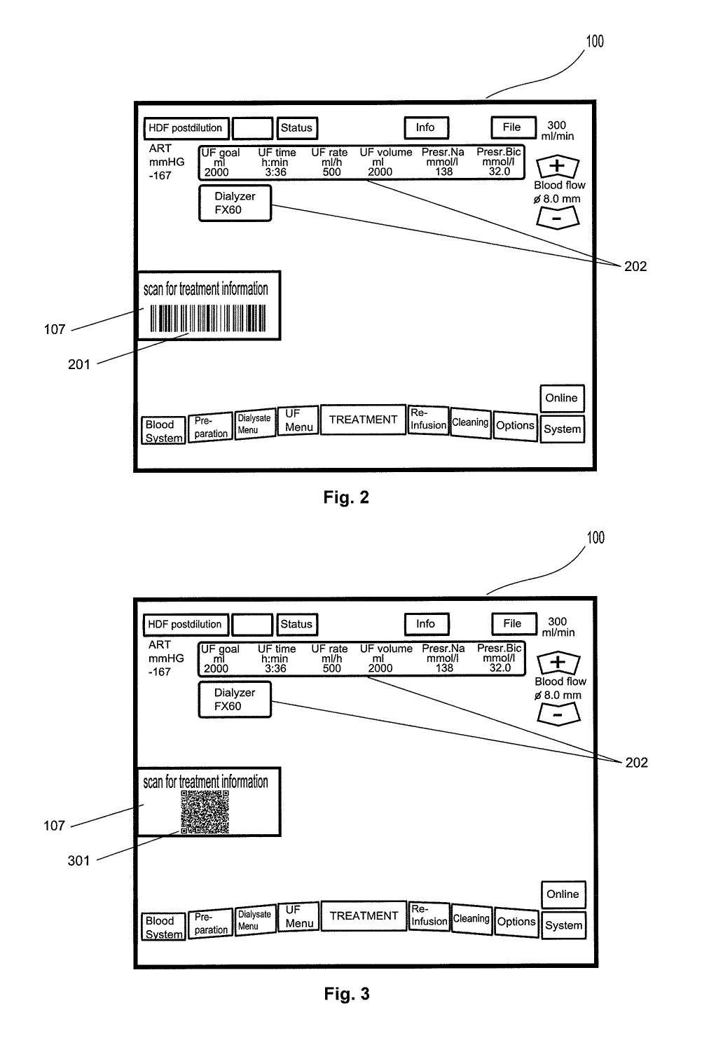

FIG. 2 shows the touchscreen display 100 in greater detail with exemplary screen contents according to the invention.

These exemplary screen contents are typical of a hemodialysis machine and illustrate the situation at the end of the treatment. The reference numeral 202 indicates information displays, which characterize and are typical of a hemodialysis treatment. These information displays provide information about the total quantities of filtrate withdrawn, the dialysis filter used or the duration of treatment, for example.

In FIGS. 2 and 3 the treatment code can be recognized by the fact that the values for the current quantity of ultrafiltrate (UF volume) match the values for the displayed values for the ultrafiltration goal (UF goal).

A graphic code which is embodied as a barcode 201 in FIG. 2 and as a QR code 301 in FIG. 3 is displayed in field 107. In addition all graphic codes that can be displayed are conceivable. In the sense of the invention, it is essential that the displayed graphic code codes for parameters of the current display treatment.

Another embodiment of the invention not shown in FIGS. 2 and 3 provides for the graphic code that is displayed to relate to the unlocking of software options.

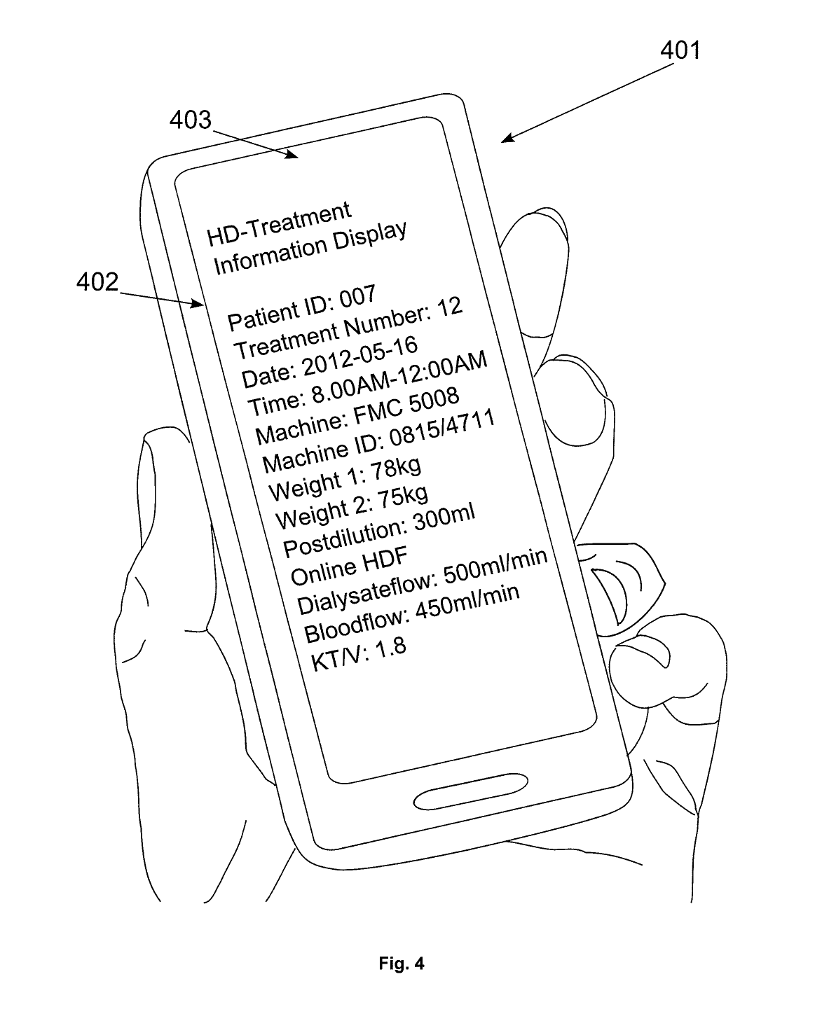

FIG. 4 portable device 401 embodied as a smartphone, information coded by the graphic code 201 or 301 being displayed on its display device 403 as an example. The operator has previously entered the code 201 or 301 on the input device of the device 401. Such an input device is typically a camera (not shown in FIG. 4). Most smartphones today are equipped with at least one camera.

Instead of a smartphone, the portable device may also be a tablet PC or a device designed specifically for this purpose.

The control unit of the portable device 401 decodes the information coded by the graphic codes 201 or 301 input and displays them on the display using software provided for this purpose. Thus there is further processing of the information encoded by the graphic code. This further processing of the information may also include storage of the information, sending the information by data transmission, for example, as email or graphic display of information pertaining to the course of the treatment in addition to mere display of the information. The possibilities of further processing of the information are also as varied as permitted by the equipment of the portable device 401 in conjunction with the software installed in it.

The information shown in FIG. 4 as an example relates to a patient who is identified by a patient number (patient ID in FIG. 4) as well as the circumstances of the treatment such as the date, time of treatment, type and serial number of the dialysis machine (machine, machine ID). In addition, certain treatment-specific information is displayed such as the weight of the patient at the start of the treatment (weight 1) and the weight at the end of the treatment (weight 2), the type of treatment (HDF corresponding to hemodiafiltration) with postdilution, dialysate flow rate (dialysate flow), blood flow rate (blood flow) and dialysis dose (KTN). The dialysis dose is of crucial importance for the efficacy of a dialysis treatment and is comprised of the product of the clearance K of the dialysis filter, quantified using the urea distribution volume V of the patient, and time T that has elapsed. DE 10 2006 032 926 by the present applicant provides additional information in this regard.

All such information displayed can be summarized as information pertaining to the medical device.

The information shown in FIG. 4 is only an example of a treatment performed using a hemodialysis machine. Any other information pertaining to the respective medical device may also be displayed.

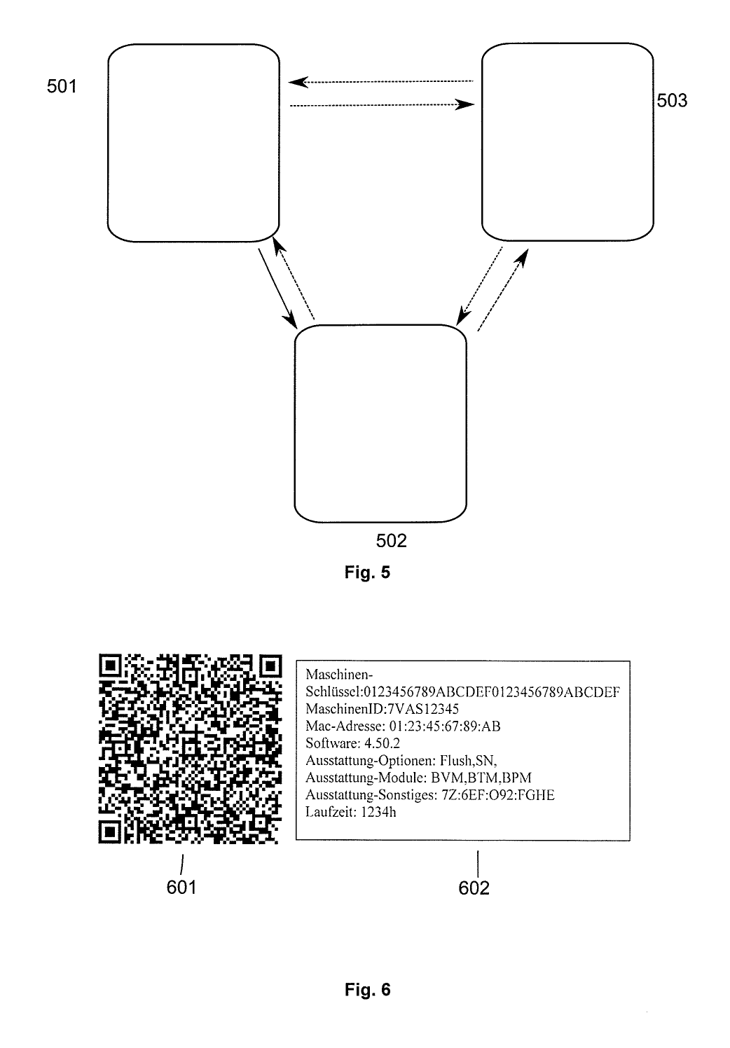

FIG. 5 shows schematically a system consisting of a medical device 501, a portable device 502 and a remote third device 503 and this illustrates the process according to the invention for unlocking the software.

The arrows shown in FIG. 5 symbolize the data transfer from one device to another device, such that the data transmission pathways that are required in the sense of the invention are characterized by solid-line arrows, and optional data transmission pathways in the sense of the invention are characterized by interrupted arrows.

As already described above, for unlocking software it is necessary to transmit an alphanumeric code which is displayed by the medical device 501 to a third remote device 503, for example, a server to which the software provider has access.

This is done according to the invention by the fact that the medical device displays a graphic code instead of the alphanumeric code which a user would otherwise have to write down. This code is entered by a portable device 502, for example, a smartphone with a camera as the input device in a manner already described above. The data transmission is represented by the solid line arrow from the medical device 501 to the portable device 502 in FIG. 5. Further processing of the input information takes place in the portable device 502. This can include display of the alphanumeric code decoded from the graphic code. In addition, the decoded code can also be sent to a third remote device 503 to which the software provider has access. This is done via the conventional data transmission pathways, for example, as email. This data transmission is indicated in FIG. 5 by the interrupted arrow from the portable device 502 to the third remote device 503. The third remote device usually has at least one send-and-receive device, wherein all devices with which data, in particular digital data, can be sent to a remote device and/or received from a remote device may be understood to include all devices with which data, in particular digital data can be sent to a remote device and/or received from a remote device. These include in particular digital interfaces, such as network interfaces which may be hardwired (for example, RJ45) or wireless (for example, WLAN, Bluetooth, infrared or UMTS). Furthermore, the third remote device has a control unit with which further processing of information, in particular for the processing of received data is possible and which controls the remote device. The control unit of the third remote device may be software-programmed.

The software provider having access to the remove device 503 may check accordingly on whether the prerequisites for unlocking the software option are met. If the outcome is positive, the software provider transmits a characteristic unlocking key, which is necessary for unlocking the software option in the medical device. This unlocking key may be transmitted to the medical device 501 in any desired manner. This includes in particular direct transmission, represented by the interrupted arrow from the remote device 503 to the medical device 501 when the devices are equipped for such a data transmission.

Frequently, however, the medical device 501 in particular is not equipped for such a data transmission, for example, when it does not have the corresponding data transmission interface such as a network connection.

Alternatively, the unlocking key can be transmitted from the remote device to the portable device, which is indicated in FIG. 5 by the broken arrow from the remote device 503 to the portable device 502, where the unlocking key can be displayed and entered manually into the medical device 501 by a user at an input device, in particular the touchscreen display of the medical device 501. However, this manual input is inconvenient and is subject to errors. Alternatively, data may be transmitted electronically from the portable device 502 to the medical device. This can be done, for example, by a USB data connection from the portable device 502 to the medical device 501 or, where this option is not available due to lack of equipment, by the portable device itself generating a graphic code which encodes the alphanumeric unlocking key and can be entered at the medical device via an input device such as a camera and decoded there.

This aspect of the invention is thus manifested in particular where the medical device does not have a network interface, for example, for reasons of data security.

The system shown in FIG. 5 is similarly used when the graphic code generated by the medical device relates to the equipment or the condition of the device.

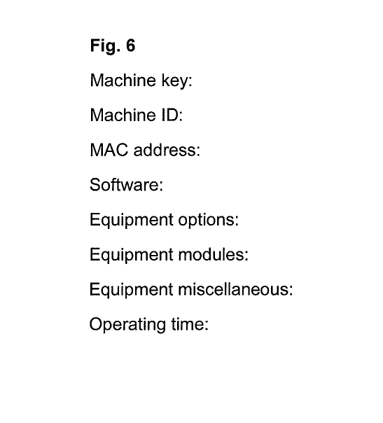

FIG. 6 shows a corresponding example of a graphic code 601, embodied here as a QR code, and an example of an equipment and state list 602, which is encoded by the code 601.

This equipment and state list 602 includes the identifications for a machine key, for example, the machine identification number, the MAC address of the control unit of the medical device, the software version, equipment options plus installed equipment modules, other equipment and the operating time of the medical device.

In addition, error messages can be encoded and forwarded in this way.

This information may be transmitted to a portable device 502 and/or to a third remote device 503 as already explained in the description of FIG. 5 and then used as information for the service technician in a service event. This service technician can have an image of the error incident and/or the concrete embodiment of a medical device in advance based on the information transmitted to him and thus be accurately prepared for the service operation accordingly and can bring along suitable replacement parts to the medical device, for example.

1. A method for display of information pertaining to a medical device, comprising the steps:
generating a graphic code which is characteristic of the information pertaining to the medical device,
displaying the graphic code.

2. The method according to claim 1, wherein the graphic code is a barcode or a QR code.

3. The method according to claim 1, wherein the information is treatment-related data.

4. The method according to claim 1, wherein the information pertains to the unlocking key of software or software options.

5. The method according to claim 1, wherein the information pertains to the equipment or the status of the medical device.

6. The method according to claim 1, wherein the information displayed is entered by a user with a portable device for further processing.

7. The method according to claim 6, wherein the further processing comprises:
sending the information to a remote device by the portable device,
generating an unlocking key from the received information in the remote device.

8. The method according to claim 7, wherein the unlocking key is transmitted to the portable device and/or to the medical device.

9. A medical device having a display device and a control unit, wherein the control unit is equipped to generate a graphic code which is characteristic of information pertaining to the medical device and to display it on the display device.

10. The medical device according to claim 9, wherein the graphic code is a barcode or a QR code.

11. The medical device according to claim 9, wherein the information is treatment data.

12. The medical device according to claim 9, wherein the information pertains to unlocking software or software options.

13. The medical device according to claim 9, wherein the information pertains to the equipment or the status of the medical device.

14. A system of a medical device according to claim 9 and a portable device having a control unit, a transmission and reception device and an input device, wherein the control unit is equipped to enter the information input with the input device and to send it by the transmission device to a remote device.

15. The system according claim 14, additionally comprising the remote device, wherein the remote device comprises a control unit, a transmission and reception device and wherein the control device is equipped to generate an unlocking key by incorporating the information received by the reception device and to send this unlocking key to the portable device and/or to the medical device via the transmission device.

16. A device according to claim 9, wherein the medical device is a blood treatment machine that is equipped for in particular for hemodialysis, for hemofiltration, for hemodiafiltration, for plasmapheresis or for automatic peritoneal dialysis.
